(19) United States
(12) Patent Application Publication
(10) Pub. No.: US 2007/0027537 A1
(43) Pub. Date: Feb. 1, 2007

(54) Method and intra-sclera implant for treatment of glaucoma and presbyopia

(76) Inventor: David Castillejos, San Diego, CA (US)

Correspondence Address: DONN K. HARMS;PATENT & TRADEMARK LAW CENTER, SUITE 100, 12702 VIA CORTINA, DEL MAR, CA, 92014, US

(21) Appl. No.: 11/528,990

(22) Filed: Sep. 27, 2006

Related U.S. Application Data
(63) Continuation-in-part of application No. 10/211,197, filed on Aug. 2, 2002.

(51) Int. Cl. A61F 2/14

(52) U.S. Cl. 623/4.1; 424/428

(57) ABSTRACT

An apparatus and method for treating presbyopia and lowering intraocular pressure employing an intra-scleral implant into an elongated cavity oriented in the radial direction of the eye. The implant has a planar portion with a longitudinal axis running therethrough and a pair of extension portions extending a distance away from said planar portion and said longitudinal axis. The implant is implanted in an incision in four quadrants of the sclera. The incisions are shaped similar to the implants. The projecting extensions into side projections of the incisions, provide an anchor to maintain the implant in the sclera.

This application is a Continuation-in-Part of U.S. application Ser. No. 10/211,197 filed Aug. 2, 2002 claiming the benefit of U.S. Provisional Application No. 60/210,227 filed Aug. 3, 2001.

### BACKGROUND OF INVENTION

1. Field of Invention

The disclosed device relates to a scleral implant. More particularly, it relates to a device which is implanted in the sclera of the eye for the treatment of excess intraocular pressure which frequently accompanies glaucoma and for the treatment of presbyopia or loss of accommodation of the eye.

Glaucoma is an eye disease wherein the patient gradually loses sight. Such vision loss is caused by damage to the optic nerve which acts like an electric cable and communicates images from the eye to the brain. High intraocular pressure frequently accompanies glaucoma and is one of the main causes of the nerve damage causing this vision loss. It is thought that increased intraocular pressure is caused when the eye's drainage canals become clogged over time. The intraocular pressure rises to levels causing damage because the correct amount of fluid cannot drain out of the eye in the normal fashion. If this excess intraocular pressure is not detected and treated, it can cause a gradual loss of vision. Such a vision loss in some cases occurs over a long period of time. However, in some cases of glaucoma the eye pressure usually rises very fast. It is thought that this happens when the eye drainage canals are blocked or covered over like the clog in a sink when something is covering the drain.

Drugs are frequently used on cases where intraocular pressure slowly builds and they frequently work well. In patients suffering a rapid rise in such pressure or a long term rise that has reached a dangerous plateau, severe eye damage and permanent loss of sight can result.

Surgery has also been used more recently to treat intraocular pressure. Clinical investigators have noted in recent years that intraocular pressure is lowered following radial incisions in the anterior sclera, known as an anterior ciliary sclerotomy. Unfortunately, for patients undergoing such a procedure, the beneficial effects are negated over a period of time following the procedure as the incisions heal and scar. Consequently, the potential for eyesight loss arises as pressure again builds following the surgery.

Another sight related problem affecting patients is that of presbyopia, which is a vision condition in which the eye and lacks an anchoring means to hold the implants in proper position in the sclera over the long term which can result in shifting of the implant reducing or eliminating its effectiveness. Further, the use of tunnels smaller than the implant tends to cause broken implants. Schachar also lacks a drug delivery means from the implant. Still further, actual dismounting of the implant can occur which would require removal from the eye, especially if it pierces the outside surface of the eye when shifting in position. Additionally, the circumferential placement of the implants is not as effective at encouraging internal drainage and reduction of intraocular pressure.

U.S. Pat. No. 6,102,045 (Nordquist) discloses a method and apparatus for lowering intraocular pressure of the eye. However, Nordquist is a filtering implant which extends into the anterior chamber of the eye through an opening in the limbus cornea. Nordquist lacks the ability to correct presbyopia that a sclera-mounted device provides and because of its delicate positioning and communication directly with the anterior chamber, Nordquist is harder to position correctly. It also lacks the ability to infuse drugs to the eye and the provision of direct communication between the anterior chamber crystalline lens of a patient's eye loses its flexibility. This loss of flexibility makes it difficult for a person to focus on close objects. While presbyopia may seem to occur suddenly once the patient discovers the problem, it is generally accepted that the cause of the sight loss is actual loss of flexibility of the lens which takes place over a number of years and usually becomes noticeable in the early to mid-forties.

Treatment to help a patient compensate for presbyopia includes prescription reading glasses, bifocals, contact lenses, and laser surgery. However such corrective lenses can be inconvenient to the wearer and laser surgery to the cornea of the eye carries with it the inherent risk to the eyesight itself if a mistake is made.

Still further, many diseases that attack the eye and eyesight require the long term administration of drugs to maintain eyesight. It is desirable to provide an easily placed device that would provide long term modulated direct communication of drugs into the eye concurrently with helping correct the internal pressure and possible vision problems of the patient.

Therefore, there is a continuing need for a medical treatment that would a require simple surgical procedure that would have long-lasting effects to relieve internal eye pressure and for the correction of presbyopia to eliminate or reduce the need for prescription lenses and without risky surgery on the lens of the eye itself. Such a treatment would be further enhanced by the provision of a drug delivery system that can be modulated for dose and time that would aid in internal pressure relief as well as other eye ailments requiring precision or long term delivery of drugs.

2. Prior Art

Surgical procedures and implantable devices have recently been developed to address the presbyopia.

U.S. Pat. No. 6,280,468 (Schachar) discloses a scleral prosthesis for treatment of presbyopia and other eye disorders. Schachar teaches the placement of a prostheses in a plurality of pockets slightly smaller than the implant, circumferentially around the pupil, to exert an outward pressure on the sclera, thereby restoring the working distance of the ciliary muscle allowing the patient relief from presbyopia. However, Schachar is oriented circumferentially around the pupil or front of the and the exterior regions of the eye increases the risk of infection to the anterior chamber.

U.S. Pat. No. 6,079,417 (Fugo) discloses a method and device for reshaping the cornea to change its topography. However, Fugo lacks the ability to increase the drainage from the eye interior to lower intraocular pressure. Fugo also is designed to mount directly into the cornea layer of the eye.

U.S. Pat. No. 5,178,604 (Baerveldt) teach the use of an implant for increasing eye drainage and reduce pressure caused by glaucoma. However, Baerveldt is simply a tube which communicates directly with the interior chamber of the eye and offers no aid to rectifying presbyopia.

As such, there is a continuing need for a reliable operative method and prostheses that will aid physicians in interrupting the relentless cycle that results in vision loss and eye damage to patients suffering from building intraocular pressure in the eye. Such a device should be insertable into the eye in a relatively easy procedure for a trained surgeon. Such a device and procedure should avoid the more delicate structures of the eye and should also avoid communicating internal eye structures directly with the exterior of the eye to prevent infection. Such a device would provide additional utility by through the optional ability to provide a drug delivery system from the implant directly to the eye. Still further, the device implanted by this method should be dimensioned with an anchor structure to insure that the implant stays properly positioned in perpetuity thereby alleviating the need for replacement or removal caused by dislocatable implants and maintaining a fixed correction of vision.

### SUMMARY OF THE INVENTION

The above problems, and others are overcome by the herein disclosed method and intra-sclera implant for the treatment of glaucoma and presbyopia. The method of insertion of the implants requires incisions be made radially into the anterior portion of the sclera. A plurality of such incisions are made radially and only into the sclera layer, with the current best number of incisions being four, with one incision within each quadrant of the anterior scleral layer eye.

Once the incisions are made in the proper quadrants and extend properly toward the rear of the eye, one implant is positioned within the space of each of the incisions. The scleral incision is then closed by opposition or using suture or other means of closure of the incision to urge the scleral flap toward the surface of the eye from where it was detached and reattach it to the sclera.

The implant is currently best formed in a unitary construction and formed of a material that is inert when in contact with body tissue. Favored materials include one or a combination of materials from a group including hydroxiapartite, silicone, polymethylmethacrylate, acrylic, and tantalum.

The unitary body of the implant can optionally be serrated or have one or a plurality of apertures running through to contact scleral tissue and anchor it. Additionally, the body of the implant can also be impregnated with a drug which thereafter would be slowly delivered into the tissue of the eye or have an internal reservoir or coating of a slowly disbursed drug that can be modulated for dose and time frame to allow for long term delivery of medication to the eye and body of the patient, from the implant.

Accordingly, it is the object of this invention disclosed herein to provide a reliable method of surgery for the placement of implants in the sclera that is easy to accomplish for the trained surgeon.

It is another object of this invention to provide an implant that is easily insertable into the scleral layer of the eye during a surgical procedure.

It is still another object of this invention to provide such an implant that has an anchoring system to insure that the implant maintains the position intended by the surgeon implanting it.

Yet another object of this invention is the provision of a method and apparatus for eye surgery that may be used to treat presbyopia as well as rising intraocular pressure.

Still further, it is an object of this invention to provide such an implant with the option of long term drug delivery directly from the implant to the eye.

These and further objectives of this invention will be brought out in the following part of the specification, wherein detailed description is for the purpose of fully disclosing the invention without placing limitations thereon.

### BRIEF DESCRIPTION OF THE DRAWING FIGURES

The accompanying drawings which are incorporated in and form a part of this specification illustrate embodiments of the disclosed device and together with the description, serve to explain the principles of the invention.

FIG. 1 depicts the placement of a plurality of implants radially in four quadrants of the eye and the steps of the method to do so.

FIG. 2 shows the implant and its placement in the scleral layer of the eye.

FIG. 3 depicts a preferred embodiment of the implant showing anchors and optional coating.

FIG. 4 depicts another preferred embodiment of the device having an internal reservoir for holding a drug to be communicated to the exterior.

FIG. 5 depicts another preferred embodiment of the device showing anchors about the exterior.

FIG. 6 depicts another preferred embodiment of the device showing a round body and anchors extending from the surface.

FIG. 7 depicts a particularly preferred mode of the device showing a “Y” or “T” shaped embodiment of the disclosed device implanted in to similarly formed pocket in the eye.

FIGS. 8-8e are a graphic depiction of the steps of the method of implant of implants into for quadrants in the eye of a patient.

### DETAILED DESCRIPTION OF THE PREFERRED EMBODIMENTS OF THE DISCLOSED DEVICE

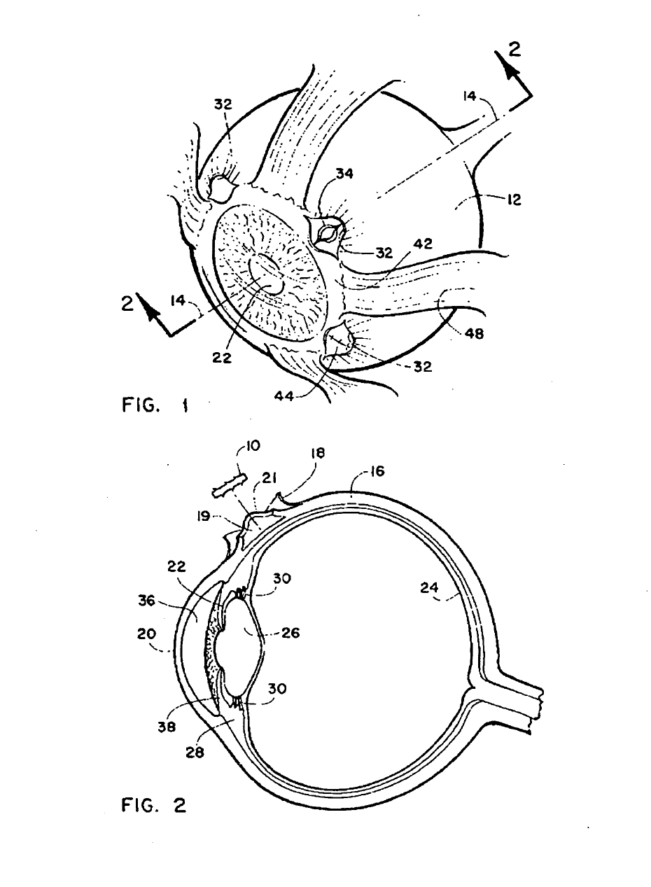

Referring now to FIGS. 1-8e depicting the preferred embodiments of the disclosed device 10, FIG. 1 depicts the preferred location and steps in the operative method for the placement of the device 10 into the eye 12. The method for surgical insertion of the implant device 10 requires incisions be made radially in the sclera 16 in relation to the cornea 22 and generally in line with the center axis 14 of the eye 12 depicted as running along line 2-2 in FIG. 1.

In its basic structure the eye 12 consists of a globe having an outer coat, a middle layer and an inner layer. The outer coat is made up of a tough fibrous, white layer—the sclera 16, which communicates with the conjunctiva 18 which is a mucous membrane that lines the inner surfaces of the eyelids and folds back to cover the front surface of the eyeball, except for the central clear portion of the outer eye which is the cornea 20. The middle layer contains pigment and forms the iris 22. The inner layer is the light seeing layer or retina 24. The lens 26 is an oval disc which sits behind the iris 22. It is conventional belief that the cornea 20 focuses approximately two-thirds of the light entering the eye 12 and the lens 26 about one third. Lens accommodation or focusing is by simple explanation accomplished by the ciliary muscle 28 pulling upon zonules 30 communicating between the ciliary muscle 28 and the lens 26.

As people age, many suffer from presbyopia which is a vision condition in which the lens 26 loses some of its flexibility, or the zonules 30 become elongated making it harder for the ciliary muscle 28 to focus the lens 26 as needed. Through implanting the device 10 using the surgical method herein disclosed, it is thought that the rejoined sclera 16 tends to pull over the device 10 and impart resulting tension to the ciliary muscle 28 giving it more working distance or travel and resulting ability to pull upon the lens 26 for better accommodation as patients receiving the device using the method of implantation have had improved vision thereafter. It is also thought that a decrease in the lens 26 equatorial diameter and a slight stretching of the zonules 30 increasing their working range also results from the scleral tension developed by the rejoining of the sclera 16 over the inserted device 10, all combine to increase amplitude of accommodation following the implantation surgery.

Intraocular pressure in the eye 12 is caused by a build up of fluid in the anterior chamber 36 and posterior chamber 38 when that fluid which is naturally produced in the eye 12 fails to be communicated through the trabecular meshwork (similar to the grate on a manhole) into the Canal of Schlemm which is the sewer system duct of the eye getting rid of excess fluid and the waste products of the eye. It has been found that following the procedure using the aforementioned method of implantation of the device 10 in the eye of patients, the drainage of aqueous fluid from the eye increases resulting in a drop of intraocular pressure. A tightening of the sclera 16 after implantation of the device 10 in the four quadrants of the eye 12, communicates a tightening or tensioning effect on the ciliary muscle 28 and its connection to the lens 26 and concurrently helps to improve the flow of fluid through the trabecular meshwork in the same region to aid in evacuation of fluid from the anterior chamber 36 and posterior chamber 38. Of course other explanations may be apparent to those skilled in the art and such are anticipated; however, in the current best mode patients do experience a drop in fluid pressure in the eye subsequent to the implantation of the device 10 using the method herein disclosed.

In exercising the surgical method for insertion of the implant device 10 the surgeon would begin with a small limited conjunctival peritomy as shown by the conjunctival incisions 32 of FIG. 1. In the current best mode of the method a plurality of conjunctival incisions 32 are performed with four being the current best number, with one in each quadrant of the eye 12 located in-between the muscles 48 attached to the exterior of the eye 12. The conjunctival incisions 32 expose the sclera 16 wherein next, in each conjunctival incision 32, a radial incision 34 is made radially or generally inline with the axis 14 of the eye 12 running through the center of the iris 22 and out the back of the eye 12. The radial incisions 34 it has been found to work well when made posteriorly 0.5 mm from the limbus and measuring substantially 3 mm in length and approximately 600 microns in depth. However, it does depend upon the dimensions of the device 10 implanted and the size of the radial incisions may change to accommodate differently dimensioned devices 10. Such a substantial inline orientation of the radial incisions 34 to the axis 14 or radial to the circle forming the iris 22 has been found to produce the best results for both accommodation and increased drainage of the eye 12.

Once the radial incisions 34 are complete and correctly axially oriented and positioned in the aforementioned manner, an implant device 10 is positioned within the space formed by the radial incision 34. At this point the radial incision 34 may be closed using a means of closure such as a suture 44 which pulls the scleral flap 21 over the implant device 10 when so rejoined exerting tension upon the sclera 16 and to communicating structures of the sclera 16. Those skilled in the art will recognize that other means of closure of such incisions are available and new means are continually being discovered and the use of such is anticipated. A radial cavity 19 is formed when the scleral flap 21 is rejoined to the sclera which surrounds the implant device 10 was placed in the radial incision. It is also anticipated that the implantation of the implant device 10 radially oriented away from the cornea 20 might be done in other fashions such as drilling or injection, or in the future, with a laser or means of mechanization, and such is anticipated. The important aspect of the device and method herein described is that the implant device 10 is placed radially oriented and surrounded by the sclera in a formed cavity and the current best mode of achieving a radial cavity 19 to hold the implant device 10 radially oriented respective to the cornea 20 is by the surgical method herein described.

Following closure of the radial incisions 34, the conjunctival incisions 32 are closed using cautery or other means of such closure. The method now being complete, the implant device 10 is properly placed to improve both the vision and fluid drainage of the patient. The implant device 10 may be removed in the reverse order.

The implant device 10 used in combination with the surgical method, in the current best mode, is formed of a material that is inert when in contact with body tissue. The implant device 10 as noted, occupies the radial cavity 19 formed when the radial incision 34 is closed in the aforementioned method. A tightening or tensioning of the sclera 16 layer is provided when the radial incision 34 is closed and the scleral flap 21 is sutured or otherwise rejoined with the sclera 16 and stretched over the implant device 10 during closure. Favored materials include one or a combination of materials from a group including hydroxiapartite, silicone, polymethylmethacrylate, acrylic, and tantalum. Those skilled in the art will recognize that other materials could be used and new materials are continually being developed for implants and the use of such is anticipated.

The implant device 10 has body portion 46 and a means to anchor the device in an elongated cavity oriented in the radial direction of the eye. It has been found as with all the embodiments of the device that the cavity works best in combination with the implants when and formed solely within the anterior scleral tissue of the eye cavity 19 to substantially prevent movement, which in a current preferred embodiment is provided by anchors 48 protruding from the body portion 46. Other means to anchor the device when placed in the radial cavity could be accomplished through the use of a serrated surface 50, or curved projections 52, or detents 54 in the exterior surface of the body 46 or apertures 56 which would communicate through the body 46. Or, one more combinations of such means to prevent movement of the implant device 10 can be used together. Optionally, should the delivery of drugs to the point of implantation be desirable, which with many illnesses such localized delivery is, the device 10 can be provided with a means to communicate drugs from a device resident supply of drugs, to the device to the surrounding eye tissue. This drug delivery system can be provided by one or a combination of micro encapsulated drug coatings or other polymer or prolonged dissolving coatings 58 on the exterior of the device, or through a reservoir 60 inside the body 46 which would hold a supply of the drug of choice in either solid or liquid form and communicate the drugs through channels 62 to the surrounding tissue. Or the material from which the device 10 is produced can be impregnated with the appropriate drug and secrete the same over time. When a reservoir 60 is used, the dosage and delivery time can be modulated by adjusting the amount of communication achieved through the channels 62, or just as the coating can, by adjusting the polymer or other substance in which the drug is dissolved to yield dissolution that will deliver the dose for amount of time desired for infusion. From the reservoir 60 the device would secrete the drugs over a determined period at the determined dose and then can be refilled through a channel 62 by a hypodermic needle 27 which would pierce the sclera 16 and refill the reservoir 60 through one of the channels 62 or a similar passage designed for such a refill. Refill can thus be accomplished without the need for the implant device 10 to be removed or disturbed from its secure mount inside the radial cavity 19.

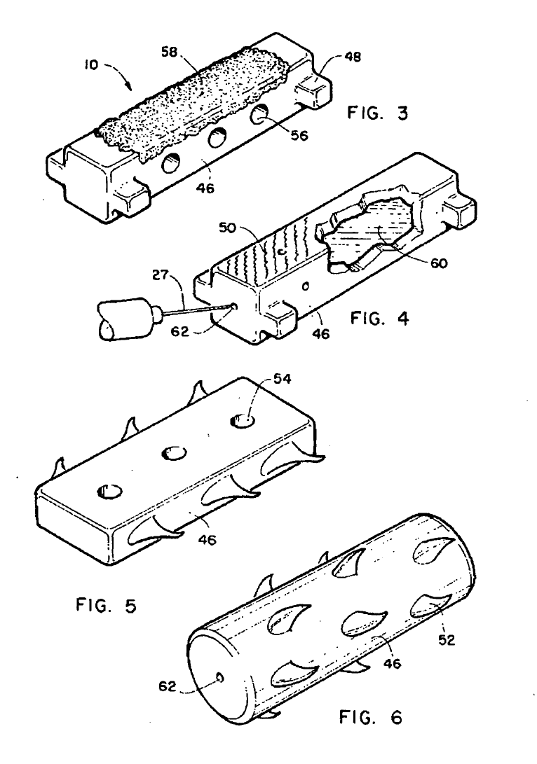

FIG. 6 depicts the device 10 with a body 46 that is round or barrel shaped rather than the cube or rectangular shape of FIGS. 3-5. The body 46 would work well in either configuration so long as one of the noted anchoring means projects from it to anchor the device 10 in the radial cavity. While the curved projections 52 are shown on all sides, it may be beneficial in some cases to omit them from one side for smooth transition of the scleral flap 21 over the implant device 10.

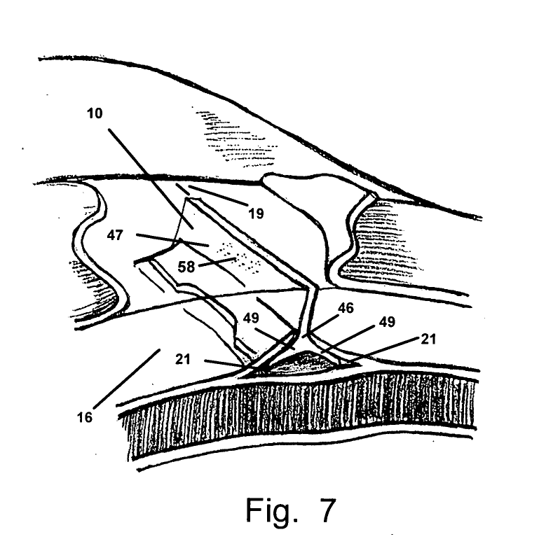

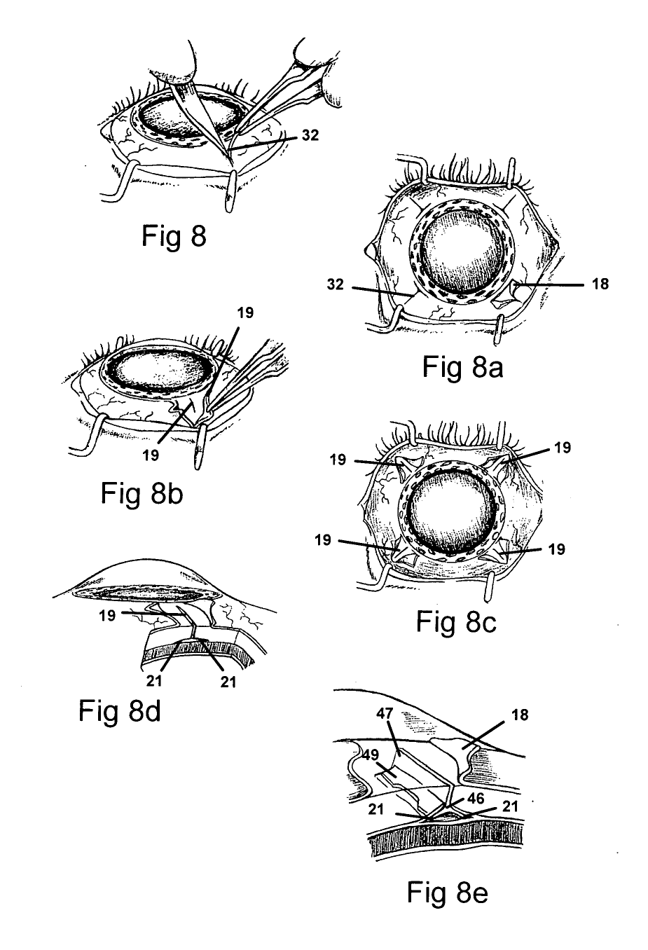

There is depicted in FIG. 7 an especially preferred mode of the device 10 which employs a “T” or “Y” similarly shaped implant device 10 as shown in FIG. 7 and 8e, which experimentation has shown to be especially effective when implanted into the sclera 16 layer of the eye using a radial incision 34 that is substantially the same shape as the implant device 10. The device 10 as shown in FIG. 7, has a body 46 with a planar component 47 having a first end, and a second end, and a longitudinal axis running through it the same as other embodiments of the device 10. From the second end of the planar component 47, two projecting extension portions, or legs 49, extend away from the axis or plane running through the planar component 47 of the body 46. The two projecting legs 49 have a length substantially equal to that of the planar component 47 and extend a distance from their communication adjacent to the second end of the planar component 47. The result is a “Y” or “T” shaped implant device 10 formed of an elongated body having the planar component 47 and two protecting legs 49. Other shapes and projecting angles and distances could be employed and are anticipated; however, the current “Y” or “T” configuration has been shown to be the easiest and most accurate for the surgeon to implant by cutting the extensions for the extending legs 49 at the bottom or the radial incision. Further, incisions so formed are predictable in their depth and have increased patient comfort and are considered the favored embodiment of the device because of both considerations.

As shown in FIG. 7, the radial incision 19 is situated as noted earlier in a radial orientation of the eye and formed in the preferred mode of the invention solely in the sclera 16. The radial incision 19 has a first portion sized to accommodate the width of the planar component 47 from the first edge closest to the conjunctiva to the second or lower edge closer to the center of the eye. At a lower edge of the radial incision 19 are formed two side incisions 21 extending from their communication with the radial incision 19 a distance to accommodate the distance dimension of extension of the two legs 49 which is the distance they extend from their respective engagements to the second end of the planar component 47. This results in a substantially “Y” or “T” shaped radial incision 19 formed into the scleral layer of the eye. All the radial incisions 19 are, of course, oriented in the radial direction of the eye and with their shape or dimension being substantially the same as the implants, provide a mount for the implants therein which will also have their longitudinal axis oriented in the radial direction of the eye once engaged in the radial incision 19.

The two legs 49 extending from their respective engagement with the second end of the planar component 47 provide a means to anchor the device in an elongated radial cavity 19 as they engage with the side incisions 21 along planes which are substantially traverse to the center portion of the radial incision 19. These legs 49 maintain the implant device 10 within the radial cavity 19 in a very secure position. Additionally, it has been found that the legs 49 provide means to impart more tension from the device 10 over a wider area and thereby enhance the resulting tension imparted to the sclera by the device 10 once implanted. This has, as such, enhanced the aforementioned utility of the device 10 to lower intraocular pressure and treat presbyopia. The surface area of the projecting legs 21 and the planar component 47 combine to provide additional and more even tensioning of the sclera once so implanted, thereby enhancing reduction of intraocular pressure and presbyopia treatment.

FIGS. 8-8e are a graphic depiction of the steps of the method of implant of implants into one or more quadrants in the eye of a patient. First in FIG. 8 and 8(a) conjunctival incisions 32 are made in the eye in a plurality which as currently noted works best with four. This is filled by the cutting of the radial incision 19 and two side incisions 21 both adapted in depth to accommodate the respective width of the planar component 47 and the distance of extension of the legs 49 from the planar component 47 on the inner edge of the implant device 10. (FIGS. 8b-8d). Finally, the implant device 10 is engaged into the radial incision 19 with the legs 49 engaged into the side incisions 21 and the planar component engaged in the vertical portion of the radial incision 19. Sealing the sclera with sutures or a flap, using this mode of the device 10 with the extending legs 49, is not required since the extending legs 49 provide means to prevent the planar component 47 from translating in the radial incision 19 out of the eye or for that matter toward the center of the eye. Instead, using the novel extending legs 49 for an anchoring means, the conjunctiva is just rejoined and the device 10 will remain implanted and resist sliding out of the sclera since the two legs 49 are engaged in the side incisions 21 which extend in opposite directions from the central incision holding the planar component 47. Tensioning imparted to the scleral layer over a wider area by both legs 49 and the planar component 47 also yield improved function of the device for both treatment of presbyopia and pressure reduction in the eye. Further, it is much more comfortable for the patient initially and later on with no need for suturing or a scleral flap to hold the device 10 in the mounted position.

As in the other modes of the device 10 noted above, this embodiment with the legs 49 extending from the planar component 47 may optionally be adapted to the delivery of drugs in the same fashion noted above wherein the device 10 is provided with a means to communicate drugs from a device-resident supply of drugs to the surrounding eye tissue. This drug delivery system can be provided by one or a combination of micro-encapsulated drug coatings or other polymer or prolonged dissolving coatings 58 on the exterior of the device as shown in the other figures, or through a reservoir 60 inside the body 46 which would hold a supply of the drug of choice in either solid or liquid form and communicate the drugs through channels 62 to the surrounding tissue. Or the material from which the device 10 is produced can be impregnated with the appropriate drug and secrete the same over time. When a reservoir 60 is used, the dosage and delivery time can be modulated by adjusting the amount of communication achieved through the channels 62 or just as the coating can, by adjusting the polymer or other substance in which the drug is dissolved to yield dissolution that will deliver the dose for amount of time desired for infusion. If a reservoir 60 is employed, it can be refilled by a hypodermic needle 27 which would pierce the sclera 16 and refill the reservoir 60 in a fashion similar to that noted on other embodiments without the need for the implant device 10 to be removed or disturbed from its secure mount inside the radially oriented cavity 19.

While all of the fundamental characteristics and features of the present invention have been described herein, with reference to particular embodiments thereof, a latitude of modification, various changes and substitutions are intended in the foregoing disclosure and it will be apparent that in some instance, some features of the invention will be employed without a corresponding use of other features without departing from the scope of the invention as set forth. It should be understood that such substitutions, modifications, and variations may be made by those skilled in the art without departing from the spirit or scope of the invention. Consequently, all such modifications and variations are included within the scope of the invention as defined by the following claims.

What is claimed is:

1. An intra-scleral implant for lowering intraocular pressure and treating presbyopia comprising:
an elongated non-dissolvable body dimensioned for implantation into an elongated cavity, said elongated cavity oriented in the radial direction of the eye and formed intrascerally in the anterior scleral tissue of the eye;
said elongated body having a planar portion have a first end, a second end;
a first side and a second side of said planar portion communicating between said first and second end;
said elongated body having a longitudinal axis running between said first end and said second end, which longitudinal axis, when implanted, is oriented in the radial direction of the eye; and
a pair of extension portions each extending a distance away from said planar portion and said longitudinal axis.

2. The intra-scleral implant of claim 1 further comprising:
each of said pair of extension portions extending a substantially equal distance away from said planar portion and said longitudinal axis.

3. The intra-scleral implant of claim 2 further comprising:
each of said pair of extension portions extending at mirrored angles from a first plane running through said planar portion, from a respective first edge adjacent to one of said first side or said second side, to a distal edge.

4. The intra-scleral implant of claim 3 further comprising:
said angles being substantially normal to said plane running through said planar portion; and
said planar portion and said extension portions forming said implant in a substantially “T” shape when viewed from said first or said second end.

5. The intra-scleral implant of claim 3 further comprising:
said extension portions extending from said planar portions at said mirrored angles to thereby dimension said implant in a substantially “Y” shape when viewed from said first or said second end.

6. The intra-scleral implant of claim 4 further comprising:
said cavity having a substantially “T” shape formed by a central cavity portion sized to accommodate said planar portion and two side cavity portions communicating with said central portion adapted to accommodate said extension portions therein.

7. The intra-scleral implant of claim 5 further comprising:
said cavity having a substantially “Y” shape formed by a central cavity portion sized to accommodate said planar portion and two side cavity portions communicating with said central portion adapted to accommodate said extension portions therein.

8. The intra-scleral implant of claim 6 further comprising:
said side cavity portions when engaged with said extension portions providing means to anchor said implant in said cavity.

9. The intra-scleral implant of claim 7 further comprising:
said side cavity portions when engaged with said extension portions providing means to anchor said implant in said cavity.

10. The intra-scleral implant of claim 8 further comprising:
said means to anchor said implant in said cavity prevents translation of said planar portion of said implant in said central cavity.

11. The intra-scleral implant of claim 9 wherein said means to anchor said body portion in said elongated cavity comprises:
said means to anchor said implant in said cavity prevents translation of said planar portion of said implant in said central cavity.

12. The intra-scleral implant of claim 8 further comprising:
means to communicate a drug dose to surrounding tissue from an implant-resident supply of a drug.

13. The intra-scleral implant of claim 9 further comprising:
means to communicate a drug dose to surrounding tissue from an implant-resident supply of a drug.

14. The intra-scleral implant of claim 12 wherein said means to communicate a drug dose to surrounding tissue from an implant-resident supply of a drug comprises:
a dissolvable coating on said intra-scleral implant.

15. The intra-scleral implant of claim 13 wherein said means to communicate a drug dose to surrounding tissue from an implant-resident supply of a drug comprises:
a dissolvable coating on said intra-scleral implant.

16. A method for lowering the intraocular pressure of an eye by implanting the intra-scleral implant of claim 1 which comprises:
making a small limited conjunctival peritomy in the conjunctiva of the eye to expose the sclera;
forming an elongated intra scleral cavity in the anterior scleral layer of the eye oriented along a first axis in the radial direction of the eye;
forming said cavity having a first having a first plane running therethrough along said first axis, and extending from a first side edge, toward the center of the eye to an inner edge;
forming two side portions of said cavity each extending a first distance away from said inner edge;
inserting said implant into said cavity with said planar portion occupying said first portion of said cavity and said a pair of extension portions each extending into said respective side portions; and
sealing said first side edge.

17. A method for treating presbyopia of an eye by implanting the intra-scleral implant of claim 1 which comprises:
making a small limited conjunctival peritomy in the conjunctiva of the eye to expose the sclera;
forming an elongated intra scleral cavity in the anterior scleral layer of the eye oriented along a first axis in the radial direction of the eye;
forming said cavity having a first having a first plane running therethrough along said first axis, and extending from a first side edge, toward the center of the eye to an inner edge;
forming two side portions of said cavity each extending a first distance away from said inner edge;
inserting said implant into said cavity with said planar portion occupying said first portion of said cavity and said a pair of extension portions each extending into said respective side portions; and
sealing said first side edge.

18. The method of claim 16 additionally comprising:
forming four of said elongated intra scleral cavities in four quadrants of the anterior sclera; and
inserting a said implant into each of said four cavities.

19. The method of claim 17 additionally comprising:
forming four of said elongated intra scleral cavities in four quadrants of the anterior sclera; and
inserting a said implant into each of said four cavities.

20. The method of claim 18 additionally comprising:
sealing said first side edge by suturing said conjunctiva of the eye.

21. The method of claim 19 additionally comprising:
sealing said first side edge by suturing said conjunctiva of the eye.

22. The intra-scleral implant of claim 2 wherein said pair of extension portions provide means to impart increased tension to the sclera over a wider surface area thereby enhancing a total tension imparted to the sclera.
